(19) United States
(12) Patent Application Publication
(10) Pub. No.: US 2015/0250574 A1
(43) Pub. Date: Sep. 10, 2015

(54) SUPPORT DEVICE USED IN MEDICAL BREAST RECONSTRUCTION

(71) Applicant: NOVUS SCIENTIFIC AB, Uppsala (SE)

(72) Inventor: Per EGNELOV, Uppsala (SE)

(73) Assignee: NOVUS SCIENTIFIC AB, Uppsala (SE)

(21) Appl. No.: 14/497,494

(22) Filed: Sep. 26, 2014

Related U.S. Application Data
(60) Provisional application No. 61/966,998, filed on Mar. 7, 2014.

(51) Int. Cl. A61F 2/00

(52) U.S. Cl. CPC A61F 2/0063; A61F 2250/0018; A61F 2230/0067

(57) ABSTRACT

A medical support device, comprising a three-dimensional mesh structure for supporting a breast implant and/or tissue, wherein the three-dimensional mesh structure comprises at least a first portion with a first value of a specific mechanical property and a second portion with a second value of said specific mechanical property, which second value of said specific mechanical property is different from the first value of said mechanical property.

### CROSS REFERENCE TO RELATED APPLICATION

This application claims priority from U.S. Provisional Patent Application No. 61/966,998, filed on Mar. 7, 2014, the entire contents of which are incorporated herein by reference.

### FIELD OF THE INVENTION

The present invention relates to a medical support device for supporting a breast implant or breast tissue, and relates in particular to a medical support device, which comprises a pre-shaped, three-dimensional, generally hemispherical, synthetic and preferably resorbable mesh structure having a number of portions with individual and specific mechanical characteristics.

### BACKGROUND OF THE INVENTION

A medical breast reconstruction is a procedure that typically involves the use of prosthetic breast implants, e.g. silicone or saline implants, which are placed either below or above the breast muscle, to recreate a female breast. In other, also common techniques, the patient's own body tissue can be used to reconstruct a breast. Within the art of reconstructive and cosmetic breast surgery it is further common to at least partly place the artificial implant or the patient's body tissue in a support device.

Examples of such support devices are disclosed in U.S. Patent Publication No. 2013/0253645 A1 to Kerr et al. In this publication, a three-dimensional medical support device is disclosed, which preferably is knitted from polymeric yarns, threads, filaments or monofilaments and which, at a given location in the device, can have an elongation of 0-30% along a horizontal axis and an elongation of 0-10% along a vertical axis, which is said to give a vertical support against gravity and the force of landing during walking and running and also to give a horizontal support which makes the implant more comfortable and allows a reconstructed breast to appear and feel more natural.

### SUMMARY OF THE INVENTION

As recognized in the prior art, support, comfort as well as appearance and feeling of a supported, reconstructed breast are important features of a medical support device for supporting a breast implant or breast tissue; and although efforts have been made to provide a support device with corresponding characteristics, there is still a need for an improved support device, which provides better support for an artificial breast implant or bodily tissue and which also stimulates integration with the existing tissue at the implant site.

A medical support device according to the present invention is intended to support and optionally also lift natural tissue or artificial material used to reconstruct a female breast structure. In one embodiment, a support device comprises a generally hemispherical, cone- or cup-shaped three-dimensional structure comprising a mesh structure, which can be made from non-biodegradable or preferably biodegradable polymeric material. In another embodiment, a support device comprises approximately one half of a generally hemispherical, cone- or cup-shaped three-dimensional structure comprising a mesh structure, which can be made from non-biodegradable or preferably biodegradable polymeric material. According to the invention, the hemispherical or semi-hemispherical three-dimensional structure is further divided into two, three or more portions with different mechanical properties. Typically, a lower portion (as seen in implanted state) of a three-dimensional mesh structure can be rather inelastic, i.e. having a high modulus of elasticity, and have high strength to provide a lot of support for the implanted breast material, while an upper portion (as seen in implanted state) of a three-dimensional mesh structure can be more elastic, i.e. have a low modulus of elasticity, and have less strength to provide more comfort for the person having a reconstructed breast.

### BRIEF DESCRIPTION OF THE DRAWINGS

FIG. 1a shows a front view and FIG. 1b shows a side view of a first embodiment of a medical support device according to the present invention, wherein the support device comprises a generally hemispherical mesh structure having three portions with different values of one or more specific mechanical properties.

FIG. 2a shows a front view and FIG. 2b shows a side view of a second embodiment of a medical support device according to the present invention, wherein the support device comprises one half of a generally hemispherical mesh structure having two portions with different values of one or more specific mechanical properties.

FIG. 3a shows a front view and FIG. 3b shows a side view of a third embodiment of a medical support device according to the present invention, wherein the support device comprises one half of a generally hemispherical mesh structure having two portions, including a inner portion and a peripheral outer portion, with different values of one or more specific mechanical properties, and wherein the support device further comprises a flange portion in the shape of an essentially flat mesh structure which is arranged outside of the hemispherical mesh structure.

FIG. 4 shows a perspective view of a fourth embodiment of a medical support device according to the present invention.

### DETAILED DESCRIPTION OF PREFERRED EMBODIMENTS

The present invention relates to a medical support device for supporting a (typically female) breast after a medical breast reconstruction surgery. A breast reconstruction surgery generally involves the re-creation of a breast either by using the patient's own tissue and/or using an artificial medical implant made from, e.g., silicone or saline.

For this purpose, the implanted material can be held in place with a three-dimensional, generally hemispherical, cup-shaped or cone-shaped support device, which can be made from degradable or non-degradable mesh material. For some medical cases, approximately one half of a generally hemispherical, cup-shaped or cone-shaped support device can be sufficient to provide the desired support for the implanted reconstruction material; and also other sizes and shapes of a three-dimensional mesh structure suitable for supporting a reconstructed breast are within the scope of the present invention.

Support devices of this kind are known in the art; and are, for example, disclosed in the above-referenced patent document. However, the inventor of the present invention has realized that the mechanical requirements regarding, e.g., strength, elasticity and stability, are not uniformly distributed over the surface of a three-dimensional, generally hemispherical support device used for supporting a reconstructed breast. Instead, typically more support is required at a lower part of the support device, whereas an upper part of the support device should be more mechanically compliant to provide the patient with as much comfort as possible and also to give the reconstructed breast a natural appearance. It can be noted that strictly speaking, terms herein like “lower” or “bottom” and “upper” or “top” refer to a medical support device in its implanted state, but the meaning should nevertheless be obvious for the person skilled in the art.

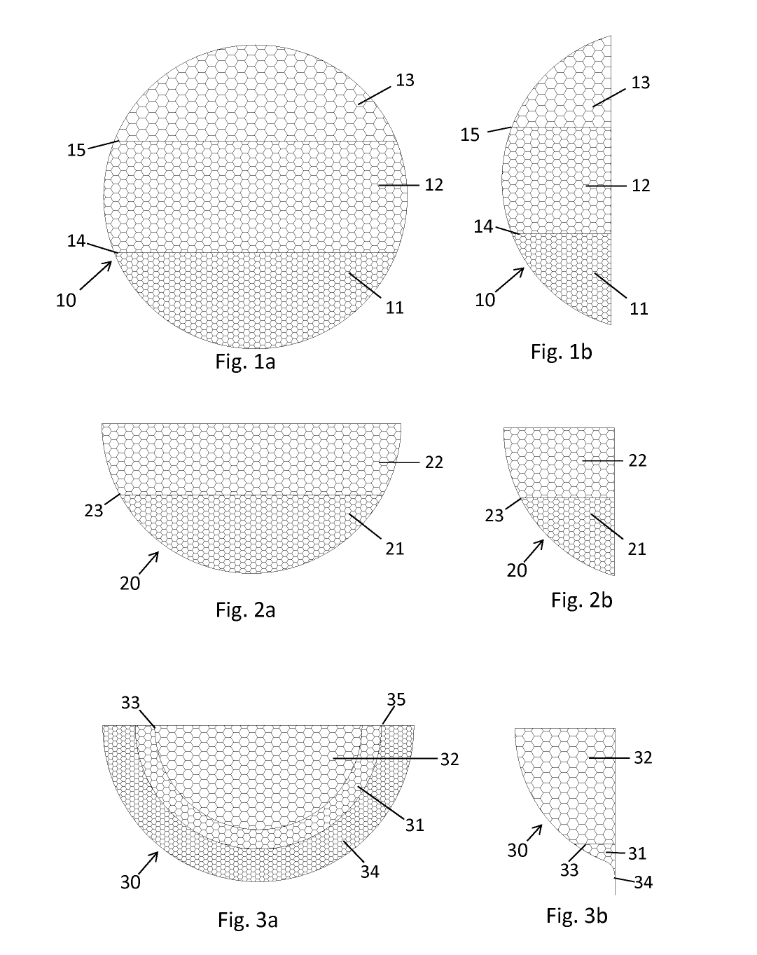

A first example of a medical support device according to the present invention is schematically depicted in FIG. 1a and FIG. 1b, where FIG. 1a shows a front view and FIG. 1b shows a side view of a support device 10 for supporting artificial and/or natural tissue used to reconstruct a female breast. The support device 10 comprises a three-dimensional, generally hemispherical, cup-shaped or cone-shaped structure 10, which preferably is a synthetic mesh structure 10 and even more preferably a biodegradable synthetic mesh structure 10, which, once implanted, degrades in a human body. Resorbable synthetic meshes of this type are, for example, the ones that are commercially available under the trade name TIGR® Matrix Surgical Mesh and are sold by the company Novus Scientific. This mesh—as well as virtually all meshes, synthetic or non-synthetic, degradable or non-degradable, that are available in the market—can with proper modifications described below be used to fulfill the requirements according to the present invention. The shape of the hemispherical structure 10 corresponds to the form of women's brassieres and could, like women's brassieres, be made in different sizes. As can be seen from FIG. 1a and FIG. 1b, the three-dimensional mesh structure 10 is further divided into three portions: a lower or bottom portion 11, an intermediate or middle portion 12, and an upper or top portion 13.

As has been indicated above, the most basic mechanical requirements on the lower portion 11 are to support and hold an implant or implanted tissue in place, which otherwise has a tendency to become dislocated, i.e. typically move downwards due to gravitation. Therefore the lower portion 11 is characterized by having a high modulus of elasticity (being rather inelastic) and/or having a high degree of stiffness, strength and/or stability. For the upper portion 13 the situation is different, since the mechanical requirements regarding elasticity, strength and stability are lower. The upper portion 13 of the mesh structure 10 should instead be comfortable for the person having a reconstructed breast and should also provide the breast with a natural and appealing overall appearance. Hence, the upper portion 13 is characterized by having a comparatively low modulus of elasticity (being rather elastic) and being compliant and/or having a low degree of stiffness, strength and/or stability. The mechanical requirements for the intermediate portion 12 are somewhere in between the mechanical requirements for the lower portion 11 and the upper portion 13; and the intermediate portion 12 is consequently characterized by having moderately high modulus of elasticity and having a moderate compliance and/or having a moderately high degree of stiffness, strength and/or stability. In this embodiment, there are three portions 11, 12 and 13 indicated in FIG. 1a and FIG. 1b. However, there could be only two portions, or more than three portions, such as four, five or more portions, all of them with different mechanical characteristics. Here it can be mentioned that partition line 14, which separates lower portion 11 and intermediate portion 12, and partition line 15, which separates intermediate portion 12 and upper portion 13, are merely introduced as guides for the eye, and have no direct physical interpretation. However, the transition from one portion to a neighbouring portion could be rather abrupt (as in FIG. 1a and FIG. 1b) or could be more gradual or even continuous such that a support device is characterized by having a high modulus of elasticity (or some other mechanical property) at a bottom rim and a low modulus of elasticity (or some other mechanical property) at a top rim, and a gradual and/or continuous transition of modulus of elasticity (or some other mechanical property) therebetween. In the latter case, an intermediate portion can be characterized by not having a fixed mechanical characteristic, but instead be characterized by having a mechanical characteristic that gradually and/or continuously transforms from the characteristics of a neighbouring lower portion to the characteristics of a neighbouring upper portion. The discussion above about number of portions as well as about transition characteristics between portions apply to all embodiments shown and described herein.

A second embodiment of a medical support device according to the present invention is disclosed in FIG. 2a and FIG. 2b, where FIG. 2a shows a front view and FIG. 2b shows a side view of a support device 20 for supporting artificial and/or natural tissue used to reconstruct a female breast. The support device 20 comprises a three-dimensional, generally half of a hemispherical, cup-shaped or cone-shaped structure 20, which preferably is a synthetic mesh structure 20 and even more preferably a biodegradable synthetic mesh structure 20, which, once implanted, degrades in a human body. As can be seen from FIG. 2a and FIG. 2b, the three-dimensional mesh structure 20 is further divided into two portions: a lower portion 21 and an upper portion 22, as indicated and visualized by partition line 23. In accordance with the teachings discussed in conjunction with the embodiment shown in FIG. 1a and FIG. 1b, the lower portion 21 is characterized by having a high modulus of elasticity (being rather inelastic) and/or having a high degree of stiffness, strength and/or stability, while the upper portion 22 is characterized by having a comparatively low modulus of elasticity (being rather elastic) and being compliant and/or having a low degree of stiffness, strength and/or stability.

A somewhat different division into portions with different mechanical characteristics is disclosed in a third embodiment of a medical support device according to the present invention. This embodiment is presented in FIG. 3a and FIG. 3b, where FIG. 3a shows a front view and FIG. 3b shows a side view of a support device 30 for supporting artificial and/or natural tissue used to reconstruct a female breast. The support device 30 comprises a three-dimensional, generally half of a hemispherical, cup-shaped or cone-shaped structure 30, which preferably is a synthetic mesh structure 30 and even more preferably a biodegradable synthetic mesh structure 30, which, once implanted, degrades in a human body. As can be seen from FIG. 3a and FIG. 3b, the three-dimensional mesh structure 30 is further divided into two portions: an outer portion 31 and an inner or central portion 32, as indicated and visualized by partition line 33. The outer portion 31 is arranged to provide support and hold an implant or implanted tissue in place, and is therefore characterized by having a high modulus of elasticity (being rather inelastic) and/or having a high degree of stiffness, strength and/or stability. The inner central portion 32 is arranged to provide comfort for the person having a reconstructed breast and should also provide the breast with a natural appearance, and is therefore characterized by having a comparatively low modulus of elasticity (being rather elastic) and being compliant and/or having a low degree of stiffness, strength and/or stability. It can in particular be noted from FIG. 3b that the inner portion 32 and the outer portion 31 are parts of the three-dimensional part of support device 30, and that outside of the three-dimensional mesh structure 30 there is another mesh structure 34 provided. The mesh structure 34 is arranged as a peripheral flange structure 34 and is arranged outside of the outer portion 31, and also outside of the three-dimensional part of medical support device 30. All embodiments envisaged herein could be provided with a similar flat flange portion. In particular, the configuration with an outer, peripheral rim or flange portion and one or more inner portions can easily be transferred to a full hemispherical mesh structure, which then comprises an inner portion with a first set of mechanical characteristics and one circumferential outer portion, which encloses the inner portion, with a second set of mechanical characteristics; or two or more outer portions with individual mechanical characteristics, e.g. a lower flange portion with a high modulus of elasticity and an upper flange portion with a low modulus of elasticity.

Different techniques can be utilized to produce a medical support device according to the present invention. For example, in knitting or weaving techniques, knitting or weaving patterns can be changed from one portion to another, e.g. by varying pore sizes or number of fibers per surface area or include more fibers with higher modulus of elasticity It is also possible to sew or otherwise join together different mesh portions having different mechanical properties, or even for some portions arrange two or more layers of mesh on top of each other. To create the three-dimensional shape, i.e. to produce the hemispherical cup-shape, meshes are commonly heat treated. This process, which in the art also is referred to as annealing, means that mesh material is exposed to heat, typically by placing the mesh material in a mould which is heated to a specific temperature. With suitable choice of material, e.g. polymeric material, the mesh material and thereby the medical support device will adopt the shape of the mould. To produce portions with different mechanical characteristics, certain areas of the mesh material can be exposed to more heat, i.e. higher temperature or longer dwell time in the mould. For example, with reference back to the third embodiment described in conjunction with FIG. 3a and FIG. 3b, higher temperature and/or longer dwell time can be provided for the outer portion 31 than for the inner central portion 32, and even higher temperature and/or long dwell time can be provided for flange portion 34, to thereby create a medical support device having a high modulus of elasticity at its rim and a low modulus of elasticity in its central portion. The temperature over the mould can be varied, for example such that a heat gradient profile is maintained which gives the mesh material a sharp, gradual or continuous transition from one part of the mesh structure to another part of the mesh structure. A lot of mechanical properties of a mesh structure can be modified, including, but not limited to, modulus of elasticity, compliance, stiffness, yield strength, tensile strength, elongation, or extension. In certain cases, a mechanical property may be directionally dependent. For example, the elongation at one location may be different in different directions. As an example, the elongation in the warp direction may be different than the elongation in the weft direction at the same location. In such a situation, the “specific mechanical property” is not merely elongation, but instead is elongation in a specified direction (for example, the elongation in the warp direction or the elongation in the weft direction).

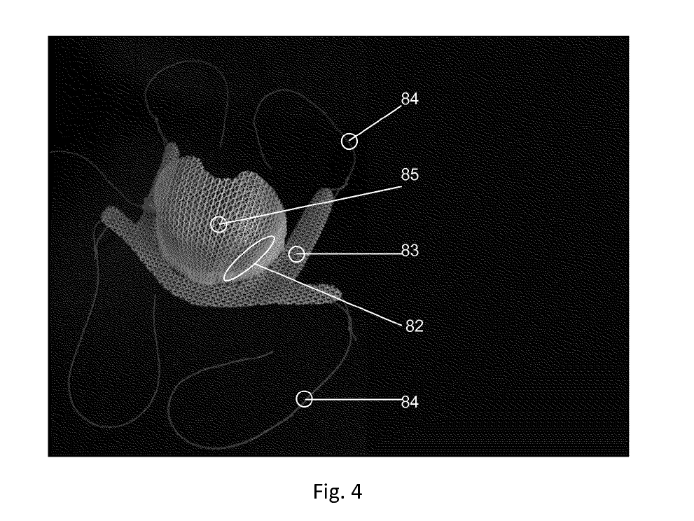

FIG. 4 shows another embodiment of a pre-shaped support device according to the present invention. The support device is preferably made from a resorbable mesh material, e.g. a mesh material like the mesh materials described in U.S. Pat. Nos. 8,016,841 and 8,313,499. The entire contents of these patents are incorporated herein by reference for the materials, manufacturing processes and other information disclosed therein relating to meshes. The material has been annealed, i.e. heat treated, to a desired, generally breast or cup shaped form. At critical areas thereof, for example at area 82 in FIG. 4 (and/or where bands or strips 83 are joined to the cup shaped portion of the device), the support device is reinforced.

The bands or stripes 83 are used for fastening of the support device to soft tissue at the implant site. Sutures, e.g. degradable sutures, can be used to attach the bands or stripes 83 to the soft tissue. Once fastened, the support device can act to lift the tissue which it supports. The support device is further provided with a set of strings 84, which are used to adjust the shape and size of the support device, to match the size of the patient's natural breast size, or according to the patient's desire.

For the device illustrated in FIG. 4, area 85, which may require a stable and rather fixed shape, can be exposed to a large amount of heat during manufacture, whereas other areas, which preferably are more elastic and compliant to match the characteristics of the tissue at the implantation site, are exposed to a smaller amount of heat.

As indicated above, one mechanical property that can characterize a mesh structure according to the invention is extension. Extension is preferably measured using the

ASTMD D3787-1 guideline for ball burst strength, with some preset parameters, such as a preload of 0.1 N and a test speed of 75 mm/min. As a non-limiting example, the extension (travel distance of the ball) measured at a load of 50 N can for a bottom or lower portion, which is characterized by having a high modulus of elasticity, a high degree of stiffness, strength and/or stability, be in the interval of 2 mm to 10 mm, while an upper or top portion, which is characterized by having a comparatively low modulus of elasticity and being mechanically compliant and/or having a low degree of stiffness, strength and/or stability, be in the interval of 5 mm to 15 mm. Intermediate portions of a mesh structure can have values between these two intervals.

Although the present invention has been described with reference to specific embodiments, also shown in the appended drawings, it will be apparent to those skilled in the art that many variations and modifications can be done within the scope of the invention as described in the specification and defined with reference to the claims below.

1. A medical support device, comprising:
a three-dimensional mesh structure configured to support a breast implant and/or tissue, wherein the three-dimensional mesh structure comprises at least a first portion, which has a first value of a specific mechanical property, and a second portion, which has a second value of said specific mechanical property, which second value of said specific mechanical property is different from the first value of said specific mechanical property.

2. A medical support device according to claim 1, wherein the three-dimensional mesh structure has a curvature corresponding to a generally hemispherical shape, or a part thereof

3. A medical support device according to claim 1, wherein said specific mechanical property is at least one of modulus of elasticity, compliance, stiffness, yield strength, tensile strength, elongation or extension.

4. A medical support device according to claim 1, wherein the three-dimensional mesh structure has a lower portion, which has the first value of said specific mechanical property, and an upper portion, which has the second value of said specific mechanical property.

5. A medical support device according to claim 1, wherein the three-dimensional mesh structure has an outer portion, which has the first value of said specific mechanical property, and an inner portion, which has the second value of said specific mechanical property.

6. A medical support device according to claim 1, wherein the three-dimensional mesh structure has a lower portion, which has the first value of said specific mechanical property, a middle portion, which has the second value of said specific mechanical property, and an upper portion, which has a third value of said specific mechanical property, which third value of said specific mechanical property is different from both the first value and the second value of said specific mechanical property.

7. A medical support device according to claim 1, wherein the medical support device further comprises an outer flange portion, which includes an essentially flat mesh structure and is arranged outside of the three-dimensional mesh structure.
